(19) United States
(12) Patent Application Publication
(10) Pub. No.: US 2019/0307990 A1
(43) Pub. Date: Oct. 10, 2019

(54) GUIDEWIRE SECUREMENT DEVICE

(71) Applicant: EndoGrip, LLC, Orem, UT (US)

(72) Inventors: Benjamin R. Fogg, Orem, UT (US); Amir A. Ghaffarian, Seattle, WA (US); Farshad Mogharrabi, Salt Lake City, UT (US)

(21) Appl. No.: 16/378,064

(22) Filed: Apr. 8, 2019

Related U.S. Application Data
(60) Provisional application No. 62/654,439, filed on Apr. 8, 2018.

(51) Int. Cl. A61M 25/09; A61M 25/02; A61M 25/01

(52) U.S. Cl. CPC A61M 25/09041; A61M 2025/024; A61M 2025/0177; A61M 25/02

(57) ABSTRACT

A guidewire securement device includes a base and an upper section. The upper section includes an aperture and a slit extending from the aperture a distance toward the base. A split in the upper section allows access to the aperture. At least the upper section is formed from a deformable material that allows for selective widening of the split for gaining greater access to the aperture and slit. A guidewire may be passed through the split and into the aperture to be secured by the device. Further passing the guidewire into the slit allows the device to grip the guidewire and limit longitudinal translation.

### CROSS-REFERENCE TO RELATED APPLICATIONS

This application claims priority to and the benefit of U.S. Provisional Patent Application Ser. No. 62/654,439, filed Apr. 8, 2018, and titled “Device to Secure Wires,” the entirety of which is incorporated herein by this reference.

### BACKGROUND

Endovascular procedures are frequently undertaken to treat conditions affecting the entire human body. Guidewires are essential to endovascular procedures. In a typical procedure, a guidewire is introduced into the human body through a small incision in the peripheral blood vessel. After the guidewire has been advanced to a targeted location within the vasculature, the guidewire then acts as a guide for larger catheters and/or other devices to reach the targeted location.

Guidewires can be difficult to manage during endovascular procedures. They are, by necessity, very small in diameter and often very long relative to the sterile surgical field within which they are intended to be used. Positioning guidewires within small vessels can be very tedious and time consuming. If the guidewire accidentally slips out of the desired position, the procedure likely has to be restarted, increasing procedure time and associated costs.

Further, if the guidewire inadvertently leaves the sterile field or comes into contact with something outside of the sterile field, it becomes contaminated and must be thrown out. For example, the end of the guidewire may slip off of an operating table and touch the floor and/or other object outside of the sterile field. This delays the procedure and requires the use of another guidewire, both of which ultimately lead to less operating room efficiency and increased costs. The risk of guidewire contamination correspondingly increases the risks of patient infection if the contamination is not noticed or properly dealt with.

A patient may also experience tissue damage from the difficulties associated with handling guidewires. For example, inadvertent movement of the guidewire can accidentally damage the patient's vasculature, potentially causing injury and risking dangerous internal bleeding.

Conventionally, guidewires may be held in place by the doctor's hand or by large wet towels. These makeshift approaches are less than ideal. If the doctor is using one hand to hold a guidewire within the sterile field, the doctor is limited to his/her other hand for performing the other tasks of the procedure. The use of large, wet towels or other such covers introduces additional large components into an already crowded surgical field.

In summary, difficulties in handling guidewires pose significant problems for patients and physicians.

### SUMMARY

Endovascular procedures commonly utilize guidewires (sometimes simply referred to as “wires”) to guide catheters and/or other medical instruments (e.g., balloons, stents, grafts) to targeted anatomical locations. During such procedures, lengths of the guidewire lie freely within the sterile field, where they are prone to accidental movement, falling off the table, and/or inadvertent contact with other nearby instruments or materials.

The securement devices described herein are configured to enable effective management of wires, catheters, and/or tubing during a medical procedure. The embodiments described herein beneficially allow for wires, catheters, and/or tubing to be secured anywhere on the surgical field, and allow users to readily position the wires, catheters, and/or tubing in desired locations and orientations. The securement devices thereby minimize or prevent contamination, procedural delays, and associated costs.

In one embodiment, a securement device includes a base and an upper section connected to the base. The upper section includes an aperture, a split extending along a surface of the upper section that provides access to the aperture, and a slit adjacent to the aperture and extending from the aperture at least partially toward the base.

The upper section may be formed from an elastomer material capable of deforming to enable selective widening of the split for providing access to the aperture. For example, at least the upper section may be formed from a suitable medical-grade polymer such as rubber, silicone, other synthetic elastomer (e.g., butadiene rubber (BR), styrene-butadiene rubber (SBR), ethylene-propylene monomer (EPM), etc.), other suitable medical-grade polymers, and combinations thereof. The material may have a Shore A durometer of about 20 to about 70.

In some embodiments, a securement includes one or more arms attached to the upper section. For example, a pair of arms may be disposed opposite one another, with each arm extending in a direction transverse to the central axis of the aperture such that a downward force against the arms causes the split to widen and provide greater access to the aperture.

The base of the securement device may include an attachment mechanism for securing the device at a desired location on the surgical field. For example, the base may include an adhesive on a bottom surface and optionally a removable cover disposed over the adhesive, one or more magnets, and/or one or more clip mechanisms.

Some embodiments also include a swivel mechanism enabling the upper section to rotate relative to the base. The swivel mechanism may include one or more locking components for rotationally locking the upper section at a desired rotational orientation.

It is to be understood that both the foregoing general description and the following detailed description are exemplary and explanatory only and are intended to provide further explanation of the invention as claimed.

### BRIEF DESCRIPTION OF THE DRAWINGS

Various objects, features, characteristics and advantages of the invention will become apparent and more readily appreciated from the following description of the embodiments, taken in conjunction with the accompanying drawings and the appended claims, all of which form a part of this specification wherein like reference numerals designate corresponding parts in the various figures and wherein the various elements depicted are not necessarily drawn to scale; and wherein:

FIGS. 1 and 2 illustrate an exemplary embodiment of a securement device for securing an endovascular structure, the securement device having a base and an upper portion, and the upper portion including an aperture extending therethrough and a slit extending from the aperture toward the base;

FIGS. 3 and 4 illustrate an alternative embodiment of a securement device having a swivel mechanism for rotating the upper portion relative to the base;

FIG. 5 illustrates an arrangement of multiple securement devices and a guidewire secured by the arrangement;

FIG. 6 illustrates an arrangement of multiple securement devices in an operating environment during an endovascular procedure on a patient; and

FIGS. 7A through 7C illustrate various positions that a guidewire and/or catheter may take within the securement device.

### DETAILED DESCRIPTION

The securement devices described herein are configured to enable effective management of wires, catheters, and/or tubing during a medical procedure. The embodiments described herein beneficially allow for wires, catheters, and/or tubing to be secured anywhere on the surgical field, and allow users to readily position the wires, catheters, and/or tubing in desired locations and orientations. The securement devices thereby minimize or prevent contamination, procedural delays, and associated costs.

Although most of the examples herein are described in relation to vascular procedures, the same embodiments and principles may be readily applied in other circumstances where management of wires, catheters, and/or tubing in the surgical field would be advantageous. For example, various gastroenterology and urology procedures may also utilize the embodiments described herein.

In addition, although most of the examples described herein relate to routing and securing a guidewire within a securement device, and then passing a catheter over the guidewire, the same securement device embodiments and principles may be used for directly routing and securing other endovascular structures such as catheters (e.g., without necessarily requiring a guidewire to first be placed within a securement device), tubing (e.g., oxygen/cannula tubing, nebulizer tubing, intravenous line tubing, or tubing for perfusion, aspiration, or drainage applications), or other medical armamentarium that could benefit from more effective securement and management.

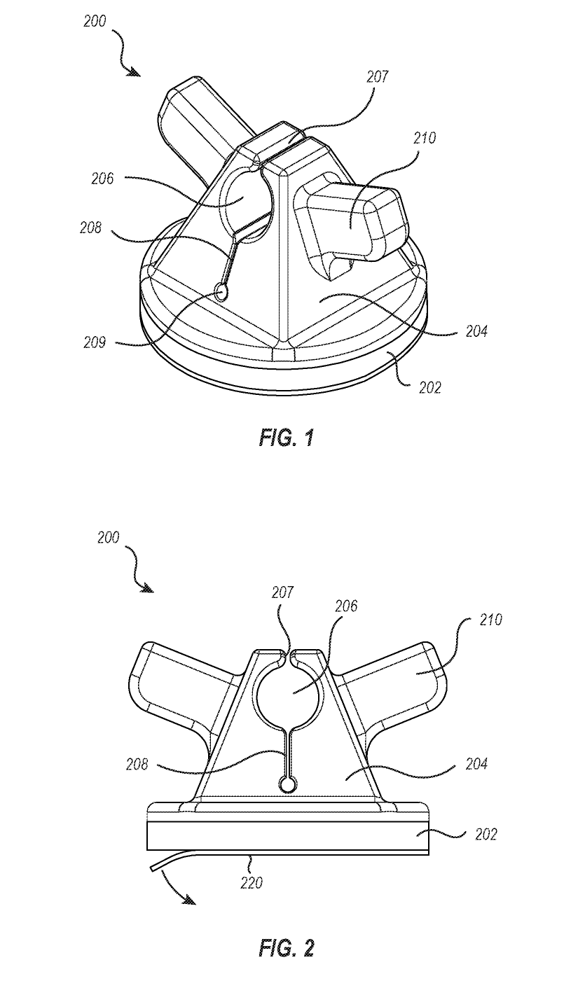

FIGS. 1 and 2 illustrate an exemplary embodiment of a securement device 200, shown in isometric view and front view, respectively. The illustrated securement device 200 includes a base 202 and an upper section 204 (which may alternatively be referred to herein as a “top”). The upper section includes an aperture 206 (which may alternatively be referred to herein as a “hollow opening” or “opening”), which is sized to allow standard sizes of wires, catheters, and/or tubing to pass therethrough.

The size of the aperture 206 may vary according to particular application needs. Typically, the aperture 206 has a diameter of about 2 mm to about 20 mm, or about 3.5 mm to about 15 mm, or about 5 mm to about 10 mm, though smaller or larger sizes may be utilized to meet particular application requirements. Although the aperture 206 is shown here as a cylindrical bore, other embodiments may have apertures of other shapes.

The illustrated securement device also includes a slit 208 disposed in the upper section 204 adjacent to the aperture 206 and extending downward therefrom toward the base 202. The slit 208 may optionally terminate in a minor aperture 209 at the bottom of the slit 208. The slit 208 allows wires to be secured and “locked” in place in a desired longitudinal position. That is, by placing the wire within the slit 208, the longitudinal translation (i.e., proximal or distal movement) of the wire relative to the securement device 200 is restricted.

As used herein, the “locked” position refers to placement of the wire within the slit 208 such that longitudinal translation of the wire is restricted. When the wire is in the aperture 206 but not the slit 208, the wire is “unlocked” but still “constrained.” That is, the wire is able to longitudinally translate due to the sufficiently large diameter and clearance of the aperture 206, but is still constrained within the aperture 206 and forced to pass through the securement device 200.

The slit 208 has a relatively narrow size to allow the sidewalls adjacent to the slit 208 to impinge against and “grip” a guidewire when it is placed within the slit 208. In some instances, the slit 208 is simply a fissure in the material that forms the upper section 204, and therefore may be completely closed until selectively moved open in response to user manipulation.

As explained in more detail below, at least the upper section 204 of the device is preferably formed of a polymer material that provides good grip of wires placed within the slit 208, such as silicone, rubber, synthetic elastomer (e.g., butadiene rubber (BR), styrene-butadiene rubber (SBR), ethylene-propylene monomer (EPM), etc.), other suitable medical-grade polymers, and combinations thereof

The material used to form at least the upper section 204 may have a Shore A durometer of about 20 to 70, or about 25 to 60, or more preferably about 30 to 50. Securement devices having durometers within the foregoing ranges have been shown to be particularly effective in gripping and securing guidewires when placed in the locked position. In a particularly preferred embodiment, at least the upper section 204 is formed from a silicone having a Shore A durometer of about 40.

The height of the upper section 204 may be varied according to particular application needs. Preferably, the upper section 204 has a height of about 10 mm to about 40 mm, or more preferably about 15 mm to about 30 mm. Heights within the foregoing ranges have been shown to allow for a slit 208 with sufficient depth to adequately hold a wire and to allow for an adequately sized aperture 206, without being excessively tall so as to form an obstacle on the surgical field or significantly disrupt the surgical field.

The upper section 204 is joined to a pair of opposing arms 210. The illustrated arms 210 join to sidewalls of the upper section 204 and extend therefrom in a direction transverse (e.g., orthogonal) to the central axis of the aperture 206. The arms 210 function to allow selective opening and closing of the aperture 206. That is, a user may manipulate the arms 210 to provide leverage for widening or narrowing the split 207 at the upper portion of the aperture 206 to thereby enlarge or constrict the aperture 206, such as for initially placing a wire, catheter, or tube within the aperture 206, for moving between a locked position within the slit 208 to an unlocked but still secured position within the aperture 206, or for removing the wire, catheter, or tube from the securement device 200.

For example, to initially place a wire within the aperture 206, a user may manipulate the arms 210 by pressing them downward toward the base 202. This forces the split 207 to widen until there is sufficient clearance to pass the wire through the split and into the aperture 206. Further manipulation of the arms 210 can also cause the aperture 206 to widen enough to open the slit 208 and allow placement of the wire within the slit 208 to thereby lock the wire. The user may then manipulate the arms 210 to bring the split 207 back to the narrower position.

In preferred embodiments, the upper section 204 is formed from a deformable/flexible polymer material that allows the securement device 200 to spring back to the illustrated default position. For example, upon release of a downward force on the arms 210, the upper section 204 will spring back to its original shape by re-narrowing the split 207 and aperture 206. Any suitable medical-grade polymer may be utilized to form the securement device 200 (or at least the upper section 204), including rubber, silicone, other synthetic elastomer (e.g., butadiene rubber (BR), styrene-butadiene rubber (SBR), ethylene-propylene monomer (EPM), etc.), other suitable medical-grade polymers, and combinations thereof

The split 207 is preferably formed on the upper side of the upper section 204 to provide ready access to the aperture 206. However, alternative embodiments may position the split 207 elsewhere, such as along a sidewall of the upper section 204. In such embodiments, the arms 210 may be correspondingly rearranged to be better positioned to function as levers for selectively opening the split 207.

The split 207 and the slit 208 are preferably substantially vertically aligned. This enables a manipulating force applied to the arms 210 to open the split 207 and the slit 208 along the same vertical line cutting through the aperture 206. However, other embodiments may offset the split 207 and the slit 208, and they need not necessarily be aligned vertically.

The arms 210 are positioned in the illustrated embodiment at an angle that is transverse to the axial direction of the aperture 206, split 207, and slit 208. This allows the split 207 and slit 208 to be opened as a result of downward force applied to the arms 210. The arms 210 may be angled upwards from the horizontal, as shown. This beneficially creates additional clearance between the arms 210 and the base 202, thereby providing more room to move the arms 210 downward for opening and gaining access to the aperture.

Alternative embodiments may position the arms 210 differently. For example, an alternative embodiment may position one or more arms at an angle substantially parallel to the axis of the aperture 206 such that lateral movement (rather than downward movement) causes the split 207 to widen.

The illustrated embodiment includes a pair of arms 210 disposed opposite one another. Some embodiments may omit arms 210, though including the arms is preferred for easier manipulation and control of the device. In some embodiments, the arms 210 are not positioned directly opposite one another. Some embodiments may include more than two arms.

In the illustrated embodiment, the slit 208 is substantially vertically-oriented. In other embodiments, the slit 208 may be offset from vertical and/or may be curved rather than straight. Similarly, although the axes of the slit 208 and the split 207 will typically be aligned with the axis of the aperture 206, in some embodiments they may be offset, so long as the split 207 is still configured to provide access to the aperture 206 and the slit 208 is still configured to receive a wire for locking the wire.

As illustrated, the upper section 204 may be wider at its base than at its top. This construction provides more material closer to the base 202 where it helps in providing structural support and integrity to the device, yet reduces the amount of material at the upper section to allow for easier bending to open the aperture 206. The illustrated embodiment has an upper section 204 with a pyramid shape having substantially planar wall surfaces, which provides for easy gripping of the device (e.g., by opposing surfaces between the thumb and finger). Other embodiments may utilize other shapes, such as cylinders, cones, truncated cones, or other shapes having curved or polygonal cross-sections.

The base 202 provides structure to enable attachment of the securement device 200 to a desired location on the surgical field. The base 202 may be circular, as shown, or alternatively may have another curved shape or a polygonal shape. The base 202 may be adapted to allow ready attachment to the surgical field. For example, the base 202 may include one or more magnets for engaging with the operating table, a railing, Mayo stand, other medical instrument, and/or corresponding magnets placed at a desired location on the surgical field (e.g., a magnetic pad).

The base 202 may additionally or alternatively include one or more clips for attachment to, for example, the patient's clothing, a surgical drape, a curtain, a railing, or other objects amenable to clips. The base 202 may additionally or alternatively include material that enables a hook/loop attachment to corresponding hook/loop material placed at a desired location in the surgical field.

The base 202 may additionally or alternatively include an adhesive for attaching to the patient's skin, the patient's clothing, the operating table, a surgical drape, or other surface amenable to an adhesive. FIG. 2 illustrates one embodiment of the securement device including a “peel-off” cover 220 on the bottom surface of the base 202. The cover 220 may be removed by the user to expose an adhesive prior to placement of the securement device 200 at a desired location.

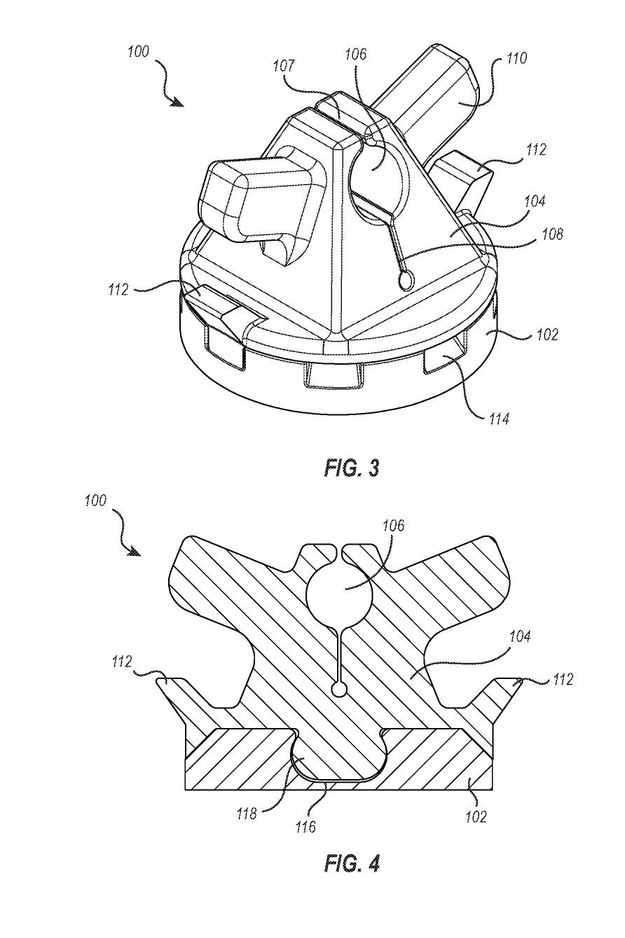

FIGS. 3 and 4 illustrate, in isometric and cross-sectional views, respectively, another embodiment of a securement device 100. The securement device 100 may share similar features of the securement device 200, and for similar components, the above description may be applied to the securement device 100, with like reference numbers indicating like components.

The securement device 100 includes a swivel mechanism that enables selective rotation of the upper section 104 relative to the base 102. As shown in the cross-sectional view of FIG. 4, the base 102 may include a socket 116 and the upper section 104 may include a corresponding ball 118. The ball 118 is received into the socket 116 to form a ball and socket joint that enables rotation of the upper section 104 relative to the base 102.

Other embodiments may include additional or alternative swivel mechanisms. For example, other embodiments may additionally or alternatively include other joint components, and/or may reverse the respective positions of the ball 118 and socket 116.

The securement device 200 may also include features for locking the rotational position of the upper section 104 relative to the base 102. In the illustrated embodiment, the upper section includes levers 112 (one or more) and the base 102 includes a plurality of corresponding notches 114 that allow for tongue and groove engagement. The levers 112 may be manipulated and flexed out of the notches 114 to allow free rotational movement of the upper section 104. When a desired rotational position is obtained, the levers 112 can be disengaged so as engage with the closest notches 114.

Other embodiments may include additional or alternative rotational locking mechanisms, such as clasps, pins, and/or other mechanical stop. Some embodiments may rely on a friction fit between the base 102 and upper section 104 that prevents inadvertent rotation yet still allows selective rotation when sufficient force is applied.

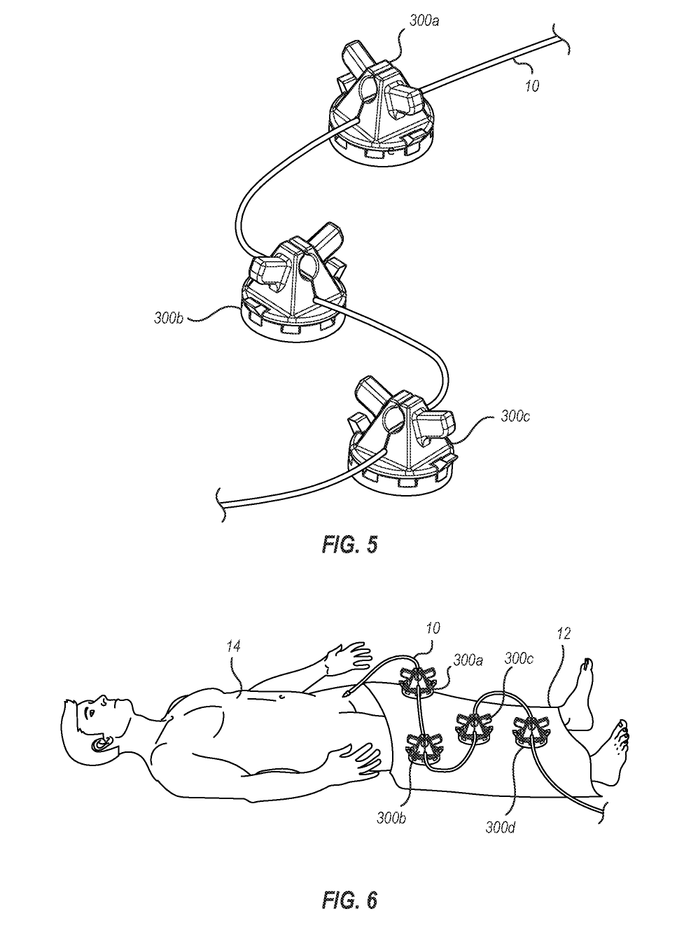

FIG. 5 illustrates a plurality of securement devices 300a, 300b, and 300c arranged to secure a guidewire 10. As shown, multiple securement devices may be arranged in a desired configuration and orientation on the surgical field to orient the guidewire 10 with desired positioning and curvature. This beneficially allows the user to control where the excess lengths of the guidewire lie on the surgical field and how the guidewire will bend and curve.

As described above, the guidewire 10 may also be locked by placing it within the slits of one or more of the securement devices 300a-300c. The enhanced control over positioning, curvature, and movement of the guidewire can beneficially reduce accidental contamination of the guidewire 10 and accidental movement of the guidewire 10, which correspondingly reduces risks to the patient related to delays in the procedure, injury, and infection.

Controlling the curvature of the guidewire 10 can also benefit caregivers and patients by allowing better alignment between the guidewire and the access point. Where a relatively long length of the guidewire remains outside the patient, bending movements can be transferred distally to portions at the access point or even within the patient, which can cause the guidewire 10 to impinge against the surrounding tissues, irritating or even potentially injuring the tissues. In contrast, the use of one or more securement devices can better ensure that the guidewire is aligned with the access point even if other sections of the guidewire are bent.

FIG. 6 illustrates an application of securement devices for managing a guidewire 10 during an endovascular procedure on patient 14. A plurality of securement devices 300a-300d may are positioned on a surgical drape 12 to manage the positioning and curvature of the guidewire 10. One or more of the securement devices 300a-300d may also receive the guidewire 10 in the locked position to selectively prevent longitudinal translation of the guidewire 10.

The embodiments described herein may be utilized to provide effective management of wires, catheters, and/or tubing in a variety of medical applications. For example, embodiments may be utilized in conjunction with interventional radiology procedures such as ablation, needle biopsies, trans jugular liver biopsy, percutaneous nephrostomy tubing, percutaneous cholecystostomy, nephron-ureteral stent placements, biliary drainage and stenting, hemodialysis access interventions, central venous access, chemoembolization therapies, angiography studies, arteriovenous malformation embolization, balloon angioplasty and stenting, tumor radiotherapy, trans jugular intrahepatic portosystemic shunting, and uterine fibroid embolization.

By way of further example, embodiments may be utilized in conjunction with interventional cardiology procedures such as cardiac catheterization procedures (e.g., angiography, percutaneous coronary angioplasty, coronary artery stent placement), pulmonary artery catheter placement, cardiac ablation procedures, cardiac pressure assessments, and pacemaker insertion.

By way of further example, embodiments may be utilized in conjunction with vascular surgery procedures such as endovascular aortic aneurysm repairs, aneurysm coiling, carotid angioplasty and stenting, peripheral vascular interventions (e.g., percutaneous transluminal arterial angioplasty and stenting), embolization of arteriovenous malformations and tumors, arteriovenous fistula interventions (thrombectomy and angioplasty), dialysis catheter placement, central and peripheral line placements, angiography studies, laser therapy, and inferior vena cava filters.

By way of further example, embodiments may be utilized in conjunction with neurosurgical procedures such as intracranial aneurysm coiling, neurovascular angioplasty and stenting, intravascular thrombolytic therapy, cerebral angiography, carotid angioplasty and stenting, brain arteriovenous malformation embolization, and sclerotherapy.

By way of further example, embodiments may be utilized in conjunction with gastroenterological procedures such as esophageal balloon dilators, stents, manometry catheters, feeding tubes, esophageal foreign-body removal, transmucosal pseudocyst drainage, colonic decompression, colonic stents, facilitating endoscope advancement, biliary duct access, pancreatic duct access, cystic access, and intrahepatic access, and biliary pancreatic cannulation.

By way of further example, embodiments may be utilized in conjunction with urological procedures such as ureteral stent placements, kidney stone retrieval, dilating urinary strictures, temporary drainage (e.g., percutaneous nephrolithotomy), and retrograde ureteroscopy.

Embodiments may also be utilized in critical care situations such as in conjunction with central or peripheral line placements.

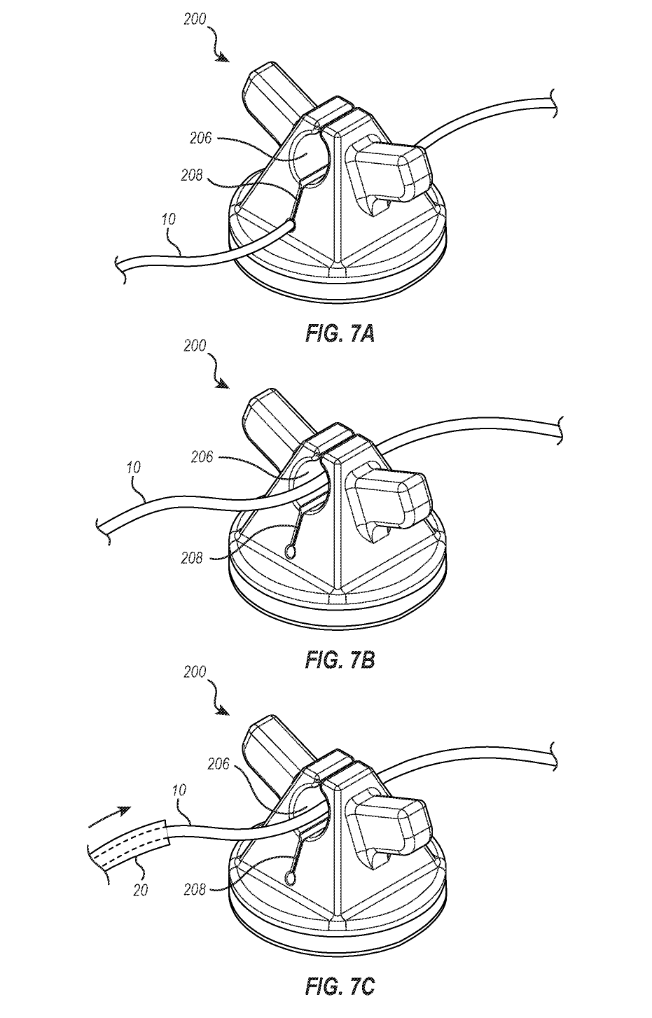

FIGS. 7A-7C illustrate various positions that endovascular structures may take within the securement device 200 during use. FIG. 7A illustrates the guidewire 10 in the locked position within the slit 208. When in the locked position, the guidewire 10 is prevented from moving proximally or distally, and this can beneficially free up a caregiver's hand that otherwise may have been required to hold the guidewire in position. It also beneficially reduces the risk of inadvertent guidewire movement, which could damage/injure the vasculature and/or remove the guidewire from the targeted anatomical location.

FIG. 7B illustrates an unlocked but still constrained position, where the guidewire 10 is positioned within the aperture 206. In this position, the guidewire 10 is free to translate longitudinally, but is still constrained within the aperture 206. The user may place the guidewire 10 in this position when translation is desired while still maintaining the overall curvature in the guidewire established by the placement of the securement device(s).

From the unlocked but still constrained position, a user may guide a catheter 20 or other medical device over the guidewire 10, as shown in FIG. 7C. The aperture 206 provides sufficient clearance to allow the catheter 20 to pass therethrough, and afterwards the securement device 200 functions to constrain the catheter 20 in the same desired configuration. The securement device 200 thus beneficially allows devices to be passed over the guidewire 10 while still constraining and holding an overall desired curvature.

The securement device 200 may also be used to longitudinally lock a larger device such as the catheter 20. For example, the catheter 20 may have a friction fit within the aperture 206, or may be forced downward at least partially into the slit 208 where it can be better gripped. Longitudinally locking the catheter 20 may be beneficial during, for example, securement of the catheter to the access site via sewing or other medically appropriate securement.

The claimed device may be embodied in other specific forms without departing from its spirit or essential characteristics. The described embodiments are to be considered in all respects only as illustrative and not restrictive. The scope of the claimed device is, therefore, indicated by the appended claims rather than by the foregoing description. All changes that come within the meaning and range of equivalency of the claims are to be embraced within their scope.

1. A securement device for securing an endovascular structure, the securement device comprising:
a base;
an upper section connected to the base, the upper section including
an aperture extending therethrough, the aperture having a central axis,
a split extending along a surface of the upper section, the split providing access to the aperture, and
a slit adjacent to the aperture and extending from the aperture at least partially toward the base.

2. The device of claim 1, wherein at least the upper section is formed from an elastomer material capable of deforming to enable selective widening of the split for providing access to the aperture.

3. The device of claim 2, wherein the device has a Shore A durometer of about 20 to about 70.

4. The device of claim 2, further comprising one or more arms attached to the upper section.

5. The device of claim 4, comprising a pair of arms disposed opposite one another, each arm extending in a direction transverse to the central axis of the aperture such that a downward force against the arms causes the split to widen.

6. The device of claim 5, wherein the arms are angled upwards from horizontal.

7. The device of claim 1, wherein the upper section is narrower at a top than at a bottom.

8. The device of claim 1, wherein the split is formed on an upper side of the upper section.

9. The device of claim 8, wherein the split and the slit are vertically aligned with one another.

10. The device of claim 1, wherein the aperture has a diameter of about 2 mm to about 20 mm.

11. The device of claim 1, wherein the upper section has a pyramid shape.

12. The device of claim 1, wherein the base includes an adhesive on a bottom surface and a removable cover disposed over the adhesive.

13. The device of claim 1, wherein the base includes one or more magnets.

14. The device of claim 1, further comprising a swivel mechanism enabling selective rotation of the upper section relative to the base.

15. A securement device for securing an endovascular structure, the securement device comprising:
a base;
an elastomer upper section connected to the base, the upper section being narrower at a top than at a bottom and including
an aperture extending therethrough, the aperture having a central axis,
a split extending along a surface of the upper section, the split providing access to the aperture, and
a slit adjacent to the aperture and extending from the aperture at least partially toward the base;
a pair of arms attached to the upper section and disposed opposite one another, each arm extending in a direction transverse to the central axis of the aperture such that a downward force against the arms causes the split to widen.

16. The device of claim 15, wherein the base includes an adhesive on a bottom surface and a removable cover disposed over the adhesive.

17. The device of claim 15, wherein the device has a Shore A durometer of about 20 to about 70.

18. A method of securing an endovascular structure on a surgical field, the method comprising:
providing one or more securement devices, each securement device being configured to receive a first endovascular structure in a locked position to limit longitudinal translation of the first endovascular structure and a constrained position that allows longitudinal translation yet maintains the first endovascular structure within the securement device;
inserting the first endovascular structure into the one or more securement devices, wherein a curvature of the first endovascular structure is at least partially defined by an arrangement of the one or more securement devices on the surgical field; and
moving the first endovascular structure between the locked position and the constrained position.

19. The method of claim 18, wherein the first endovascular structure is a guidewire.

20. The method of claim 19, further comprising passing a catheter over the guidewire and passing the catheter through at least one of the securement devices with the guidewire in the constrained position.
